(19) United States
(12) Patent Application Publication
(10) Pub. No.: US 2010/0324664 A1
(43) Pub. Date: Dec. 23, 2010

(54) Bifurcated Stent Assemblies

(76) Inventors: Asher Holzer, Haifa (IL); Eli Bar, Moshav Megadim (IL); Ofir Paz, Rishon-lezion (IL)

Correspondence Address: D. Kligler I.P. Services LTD, P.O. Box 25, Zippori, 17910, IL

(21) Appl. No.: 12/445,968

(22) PCT Filed: Oct. 18, 2007
(86) PCT No.: PCT/IL2007/001253

§ 371 Date: Apr. 17, 2009

Related U.S. Application Data
(63) Continuation-in-part of application No. 11/582,354, filed on Oct. 18, 2006.
Application No. 11/582,354 is a continuation-in-part of application No. 11/797,168, filed on May 1, 2007.
(60) Provisional application No. 60/852,392, filed on Oct. 18, 2006.
(60) Provisional application No. 60/860,485, filed on Nov. 22, 2006.
(60) Provisional application No. 60/877,162, filed on Dec. 27, 2006.

(51) Int. Cl. A61F 2/82; B23P 11/00

(52) U.S. Cl. 623/1.35; 29/428

(57) ABSTRACT

Disclosed is a stent assembly for expanding in vivo vessels, the assembly comprises: two stents, a first stent and a second stent, the two stents positioned so that a forward end of the first stent is separated by a predetermined distance from a rearward end of the second stent, and a stent jacket spanning the predetermined distance such that a first end of the jacket is operatively associated with the first stent and a second end of the jacket is operatively associated with the second stent.

This Application claims benefit of priority from U.S. patent application Ser. No. 11/797,168 filed May 1, 2007 which is a continuation-in-part of PCT Patent Application No. PCT/IB2006/051874 filed May 24, 2006, which in turn claims the benefit of U.S. Provisional Patent Applications Nos. 60/683,788 filed May 24, 2005; 60/716,100 filed Sep. 12, 2005; and 60/742,460 filed Dec. 5, 2005.

This application is also a continuation-in-part of pending U.S. patent application Ser. No. 11/582,354 filed Oct. 18, 2006.

In addition, this application claims priority from U.S. Provisional Patent Applications Nos. 60/852,392 filed Oct. 18, 2006, 60/860,485 filed Nov. 22, 2006, 60/860,486 filed Nov. 22, 2006 and 60/877,162 filed Dec. 27, 2006.

The contents of all of the above documents are incorporated by reference as if fully set forth herein.

### FIELD AND BACKGROUND OF THE INVENTION

The present invention, in some embodiments thereof, relates to stent assemblies that are deployed in bifurcated vessels and, more particularly, but not exclusively, to bifurcating stent assemblies having low bulk at vessel bifurcations between branch vessels and parent vessels.

While mono-tubular stents have resulted in improved long-term blood flow, stents are associated with severe problems when deployed in a bifurcated lumen, meaning a parent lumen from which a branch vessel splits. It is estimated that 15% to 20% of all stents are deployed at bifurcations.

Treatment of stenotic lesions at bifurcations is associated with increased early complications including compromise of either the branch vessel or the parent vessel and increased potential for restenosis.

One method for stenting a bifurcating vessel includes placing a first stent having a substantially circular side opening in a parent vessel and a second stent having a flared end for stenting the branch vessel.

The first stent is positioned in the lumen of the parent vessel and expanded, after which the second, flared stent is pressed through the side opening of the first stent and expanded in the branch vessel.

One drawback of this method is the difficulty of properly aligning the side opening of the first stent with the branch vessel bifurcation so that the branch vessel stent passes into the branch vessel. Another drawback of this system is that the second, flared, stent is difficult to position properly, and may protrude into the blood stream causing thrombosis.

Another method of treating bifurcations is called the crush method, an example of which is seen in U.S. Patent application 20050049680 (Fischell et al), the entirety of which is hereby incorporated by reference as if fully disclosed herein.

In this method, a first stent is placed into the branch vessel and expanded so that a portion of the stent protrudes into the parent vessel. A second stent is expanded in the parent vessel, crushing the protruding portion of the first stent against the parent vessel wall around the branch vessel opening.

If the first stent is not properly crushed, however, the end of the stent will protrude into the bloodstream, often resulting in thrombosis. Additionally, during crushing, the first stent may pull away from the branch vessel so that there is no support of the branch vessel where support is needed most. Finally, the crush method deposits a large amount of metal at the entrance to the branch vessel lumen, where the tissue is thin and often incapable of supporting the metallic bulk, resulting in restenosis.

### SUMMARY OF THE INVENTION

According to some embodiments of the invention there is provided a stent assembly comprising two radially expandable mesh stents separated by a distance with a common stent jacket spanning the distance therebetween.

In embodiments, the assembly is configured to be positioned so the two mesh stents are located in a parent vessel on either side of a branch vessel bifurcation and the jacket spans the lumen associated with a bifurcation.

A mesh stent in a contracted state is delivered the site and passed through an aperture in the jacket into the branch vessel and expanded. The aperture expands so that the third stent remains at least partially covered by the stent jacket and the stent jacket spanning between the first, second and third stents supports the stenotic tissue of the bifurcation therebetween.

The stents are optionally deployed using any one of several techniques, including inter alia pre dilatation angioplasty, post angioplasty, and the above noted “kissing technique” and direct dilation stenting techniques.

In other embodiments an end of the third stent, in an unexpanded state, is pressed into the jacket and the third stent is expanded, thereby stretching a portion of the stent jacket. Thereafter, the expanded jacket portion is punctured by a puncturing instrument, and an expanding balloon is expanded within the punctured portion. Thereafter, the third stent is passed into the branch vessel and expanded.

In still further embodiments, a stent assembly comprises two radially expandable mesh parent vessel stents separated by a distance with a common stent jacket spanning the distance therebetween. Third and fourth stents are transported within the lumen formed by the first and second stents and the jacket therebetween. Upon reaching an in situ location, the first and second stents are expanded and the third and fourth stents are pressed through the jacket therebetween to expand in branch vessels.

According to one aspect of the present invention, there is provided a stent assembly for expanding in vivo vessels, the assembly comprising: two stents, a first stent and a second stent, the two stents positioned so that a forward end of the first stent is separated by a predetermined distance from a rearward end of the second stent, and a stent jacket spanning the predetermined distance such that a first end of the jacket is operatively associated with the first stent and a second end of the jacket is operatively associated with the second stent.

In embodiments, upon radial expansion of the first stent and the second stent, the first end of the jacket expands radially encircles at least a portion of the first stent and the second end of the jacket expands radially and encircles at least a portion of the second stent.

In embodiments, the stent jacket spanning the predetermined distance comprises a length sufficient to longitudinally encircle an axially disposed third stent in a contracted state and axially disposed and movably set on a guide wire.

In embodiments, the stent jacket spanning the predetermined distance comprises an internal surface configured to have a cross sectional diameter sufficient to encircle the third stent while the assembly is being delivered to an in situ location.

In embodiments, the guide wire is configured with an angulated distal portion that allows manipulation proximate to a portion of the stent jacket following expansion of the first stent and the second stent at the in situ location.

In embodiments, the angulated distal portion comprises an angle to an axis running between the first stent and the second stent of at least about 15 degrees and no more than about 165 degrees.

In embodiments, the angulated distal portion of the guide wire has a constant cross-sectional diameter and the stent jacket spanning the predetermined distance comprises at least one aperture of sufficient diameter to allow passage of the cross-sectional diameter.

In embodiments, the portion of the guide wire is configured with sufficient strength to be manipulated through the aperture.

In embodiments, the at least one aperture is expandable and configured to expand to a diameter sufficient to encircle an outer surface of the third stent while the third stent in the contracted state.

In embodiments, the mean diameter of the at least one aperture is configured to further expand when the contracted third stent is expanded while encircled by the aperture.

In embodiments, at least a portion of the stent jacket spanning the predetermined distance is configured to encircle at least a portion of an outer surface of the third stent when the third stent is in an expanded state.

In embodiments, a first end of the third stent comprises a friction surface configured to catch a portion of the stent jacket when the friction surface stent is pressed against the portion while the third stent is expanding.

In embodiments, the stent jacket comprises a stretchable material configured to stretch across the first end of the stent while the third stent is expanding during the pressing.

In embodiments, following expansion of the third stent, a stretched portion of the stent jacket is configured to be punctured by a puncturing tool.

In embodiments, the stent jacket includes an intact portion spanning the predetermined distance, the intact portion configured to remain intact following the puncturing.

In embodiments, at least a portion of the intact portion includes at least one fold, the at least one fold being adhered by a pressure-sensitive self-adhering adhesive.

In embodiments, the punctured portion of the stent jacket is expandable and configured to form a mean diameter that is sufficient to allow the third stent to pass through the puncture.

In embodiments, the puncturing tool includes an expandable balloon.

In embodiments, the stent jacket spanning the predetermined distance comprises at least one aperture configured to encircle the expandable balloon in a contracted state.

In embodiments, the at least one aperture is configured to rip as the expandable balloon is inflated.

In embodiments, a portion of an outer surface of the third stent is configured to slidingly pass through the aperture following the rip and the aperture is configured to remain encircled around at least a portion of an outer surface of the third stent.

In embodiments, while the assembly is being delivered to an in situ location: a first portion of the stent jacket spanning the predetermined distance is configured to encircle the axially disposed third stent in a contracted state, and a second portion of the stent jacket spanning the predetermined distance is configured to encircle an axially disposed fourth stent in a contracted state.

In embodiments, the guide wire upon which the third stent is set comprises a first guide wire and the fourth stent is axially set on a second guide wire having an angulated distal portion comprising an angle to an axis running between the first stent and the second stent of at least about 15 degrees and no more than about 165 degrees.

The assembly according to any one of the previous claims, wherein following expansion the vessels are supported with one layer of stent metal.

In embodiments, for example for use in a coronary vessel, the first stent is positioned between at least one millimeter and not more than about 20 millimeters from the second stent.

In other embodiments, the first stent is positioned about three millimeters from the second stent. Optionally, the first stent and second stent are placed in positions that stretch the jacket therebetween.

In embodiments, upon radial expansion of the first and second stents, the first jacket end expands radially and encircles at least a portion of the first stent and the second end of the jacket expands radially and encircles at least a portion of the second stent.

In embodiments, during expansion, the first stent and the second stent are of a sufficient diameter to press at least a portion of the inner walls of a parent vessel with a pressure of at least one atmosphere and no more than about 50 atmospheres.

In embodiments, during expansion, the first stent and the second stent are of a sufficient diameter to press at least a portion of the inner walls of a parent vessel with a pressure of about 15 atmospheres.

In embodiments, during expansion, the third stent is of a sufficient diameter to press at least a portion of the inner walls of a branch vessel with a pressure of at least one atmosphere and no more than about 50 atmospheres.

In embodiments, during expansion, the third stent is of a sufficient diameter to press at least a portion of the inner walls of a branch vessel with a pressure of about 15 atmospheres.

In embodiments, during expansion, the first stent and the third stent are of a sufficient diameter to press at least a portion of the inner walls of a parent vessel with a pressure of at least one atmosphere and no more than about 50 atmospheres.

In embodiments, during expansion, the first stent and the third stent are of a sufficient diameter to press at least a portion of the inner walls of a parent vessel with a pressure of about 15 atmospheres.

In embodiments, during expansion, the second stent is of a sufficient diameter to press at least a portion of the inner walls of a branch vessel with a pressure of at least one atmosphere and no more than about 50 atmospheres.

In embodiments, during expansion, the second stent is of a sufficient diameter to press at least a portion of the inner walls of a branch vessel with a pressure of about 15 atmospheres.

In embodiments, at least a portion of the intact portion includes a pressure-sensitive self-adhering adhesive.

In embodiments, the adhesive is an adhesive from the group of adhesives comprising: fibrin, biological glue, collagen, hydrogel, hydrocolloid, collagen alginate, and methylcellulose.

In embodiments, at least a portion of the at least one fold is configured to adhere in response to pressure of at least about one atmosphere and no more than about 20 atmospheres.

In embodiments, during expansion, the first stent and the second stent are of a sufficient diameter to press at least a portion of the inner walls of a parent vessel with a pressure of at least one atmosphere and no more than about 50 atmospheres.

In embodiments, during expansion, the first stent and the second stent are of a sufficient diameter to press at least a portion of the inner walls of a parent vessel with a pressure of about 15 atmospheres.

In embodiments, during expansion, the third stent is of a sufficient diameter to press at least a portion of the inner walls of a branch vessel with a pressure of at least one atmosphere and no more than about 50 atmospheres.

In embodiments, during expansion, the third stent is of a sufficient diameter to press at least a portion of the inner walls of a branch vessel with a pressure of about 15 atmospheres.

In embodiments, during expansion, the first stent and the third stent are of a sufficient diameter to press at least a portion of the inner walls of a parent vessel with a pressure of at least one atmosphere and no more than about 50 atmospheres.

In embodiments, during expansion, the first stent and the third stent are of a sufficient diameter to press at least a portion of the inner walls of a parent vessel with a pressure of about 15 atmospheres.

In embodiments, during expansion, the second stent is of a sufficient diameter to press at least a portion of the inner walls of a branch vessel with a pressure of at least one atmosphere and no more than about 50 atmospheres.

In embodiments, during expansion, the second stent is of a sufficient diameter to press at least a portion of the inner walls of a branch vessel with a pressure of about 15 atmospheres.

In embodiments, the third stent is set at an angle to an axis passing through the first stent and the second stent of at least about 15 degrees and no more than about 165 degrees.

In embodiments, during expansion, the third stent is of a sufficient diameter to press at least a portion of the inner walls of a branch vessel with a pressure of at least one atmosphere and no more than about 50 atmospheres.

In embodiments, during expansion, the third stent is of a sufficient diameter to press at least a portion of the inner walls of a branch vessel with a pressure of about 15 atmospheres.

In embodiments, the third stent is positioned to expand substantially outward and substantially radially opposite to the expansion of the fourth stent.

In embodiments, during expansion, the fourth stent is of a sufficient diameter to press at least a portion of the inner walls of a branch vessel with a pressure of at least one atmosphere and no more than about 50 atmospheres.

In embodiments, during expansion, the fourth stent is of a sufficient diameter to press at least a portion of the inner walls of a branch vessel with a pressure of about 15 atmospheres.

In embodiments, during expansion, the first stent and the second stent are of a sufficient diameter to press at least a portion of the inner walls of a parent vessel with a pressure of at least one atmosphere and no more than about 50 atmospheres.

In embodiments, during expansion, the first stent and the second stent are of a sufficient diameter to press at least a portion of the inner walls of a parent vessel with a pressure of about 15 atmospheres.

In embodiments, the stents comprise a metallic base from the group consisting of: stainless steel, nitinol, tantalum, MP35N alloy, a cobalt-based alloy, a cobalt-chromium alloy, platinum, titanium, or other biocompatible metal alloys.

In embodiments, the stents are selected from the group consisting of: a cardiovascular stent, a coronary stent, a peripheral stent, an abdominal aortic aneurysm stent, a cerebral stent, a carotid stent, an endovascular stent, an aortic valve stent, and a pulmonary valve stent.

In embodiments, the stent jacket comprises a material manufactured by a process from the group consisting of: interlacing knitting, interlocked knitting, braiding, interlacing, and/or dipping a porous mold into one or more reagents.

In embodiments, during expansion the stents are configured to expand in a manner that dilates the adjacent lumens.

In embodiments, following expansion the lumens are supported by one layer of stent metal.

According to one aspect of the invention, there is provided a method for manufacturing a stent assembly for expanding in vivo vessel lumens, the method comprising: providing two axially aligned radially expandable mesh stents, comprising a first stent having a forward end at a predetermined distance from a rearward end of a second stent, attaching a first end of a stent jacket to the first stent, attaching a second end of the stent jacket to the second stent, such that an intermediate portion of the jacket spans the predetermined distance, and encircling a third stent in a contracted state coaxially aligned within the jacket.

In embodiments the method includes: expanding the two axially radially expandable mesh stent, and angling the third stent at an angle to an axis running between the first stent and the second stent of at least about 15 degrees and no more than about 165 degrees.

In embodiments the method includes: angling a fourth stent at an angle to an axis running between the first stent and the second stent of at least about 15 degrees and no more than about 165 degrees.

In embodiments, the radially expandable stent comprises a metallic base from the group consisting of: stainless steel, nitinol, tantalum, MP35N alloy, a cobalt-based alloy, a cobalt-chromium alloy, platinum, titanium, or other biocompatible metal alloys.

In embodiments, the radially expandable stent comprises a bio degradable/bio-absorbable base from the group consisting of: PGLA, PLLA, PLA, bio-resorbable magnesium, or other bio resorbable compounds.

In embodiments, the jacket and the stents comprise a material selected from the group consisting of: polyethylene, polyvinyl chloride, polyurethane, nylon and a biocompatible polymer fiber.

In embodiments, the jacket and the stents comprise a material selected from the group consisting of: nitinol, stainless steel shape memory materials, metals, synthetic biostable polymer, a natural polymer, and an inorganic material. In embodiments, the biostable polymer comprises a material from the group consisting of: a polyolefin, a polyurethane, a fluorinated polyolefin, a chlorinated polyolefin, a polyamide, an acrylate polymer, an acrylamide polymer, a vinyl polymer, a polyacetal, a polycarbonate, a polyether, a polyester, an aromatic polyester, a polysulfone, and a silicone rubber.

In embodiments, the natural polymer comprises a material from the group consisting of: a polyolefin, a polyurethane, a Mylar, a silicone, and a fluorinated polyolefin.

In embodiments, the jacket and the stents comprise a material having a property selected from the group consisting of: compliant, flexible, plastic, and rigid.

In embodiments, the assembly includes an active pharmaceutical ingredient.

In embodiments, the API comprises a chemotherapeutic selected from the group consisting of peptides, proteins, nucleic acids, monoclonal antibodies, L-cell agonists, super oxide dismutase Interleukin-10, glucorticoids, sulphazalazine, calcitonin, insulin, 5-fluoracil, leucovorin, fluoropyrimidine S-1,2-deoxycytidine, analgesics, antibacterials, antibiotics, antidepressants, antihistamines, antihelminths, anti-inflammatory agents, antiirritants, antilipemics, antimicrobials, antimycotics, antioxidants, antipruritics, antiseptic, antiswelling agents, antiviral agents, antiyeast agents, astringents, topical cardiovascular agents, chemotherapeutic agents, corticosteroids, fungicides, hormones, hydroxyacids, lactams, non-steroidal anti-inflammatory agents, progestins, statines, sanatives and vasodilators and mixtures thereof.

In embodiments, the API comprises an analgesic selected from the group consisting of benzocaine, butamben picrate, dibucaine, dimethisoquin, dyclonine, lidocaine, pramoxine, tetracaine, salicylates and derivatives, esters, salts and mixtures thereof.

In embodiments, the API comprises an antibiotic selected from the group consisting of amanfadine hydrochloride, amanfadine sulfate, amikacin, amikacin sulfate, aminoglycosides, amoxicillin, ampicillin, ansamycins, bacitracin, beta-lactams, candicidin, capreomycin, carbenicillin, cephalexin, cephaloridine, cephalothin, cefazolin, cephapirin, cephradine, cephaloglycin, chloramphenicols, chlorhexidine, chlorhexidine gluconate, chlorhexidine hydrochloride, chloroxine, chlorquinaldol, chlortetracycline, chlortetracycline hydrochloride, ciprofloxacin, circulin, clindamycin, clindamycin hydrochloride, clotrimazole, cloxacillin, demeclocycline, diclosxacillin, diiodohydroxyquin, doxycycline, ethambutol, ethambutol hydrochloride, erythromycin, erythromycin estolate, erythromycin stearate, farnesol, floxacillin, gentamicin, gentamicin sulfate, gramicidin, griseofulvin, haloprogin, haloquinol, hexachlorophene, iminocylcline, iodochlorhydroxyquin, kanamycin, kanamycin sulfate, lincomycin, lineomycin, lineomycin hydrochloride, macrolides, meclocycline, methacycline, methacycline hydrochloride, methenamine, methenamine hippurate, methenamine mandelate, methicillin, metronidazole, miconazole, miconazole hydrochloride, minocycline, minocycline hydrochloride, mupirocin, nafcillin, neomycin, neomycin sulfate, netilmicin, netilmicin sulfate, nitrofurazone, norfloxacin, nystatin, octopirox, oleandomycin, orcephalosporins, oxacillin, oxytetracycline, oxytetracycline hydrochloride, parachlorometa xylenol, paromomycin, paromomycin sulfate, penicillins, penicillin G, penicillin V, pentamidine, pentamidine hydrochloride, phenethicillin, polymyxins, quinolones, streptomycin sulfate, tetracycline, tobramycin, tolnaftate, triclosan, trifampin, rifamycin, rolitetracycline, spectinomycin, spiramycin, streptomycin, sulfonamide, tetracyclines, tetracycline, tobramycin, tobramycin sulfate, triclocarbon, triclosan, trimethoprim-sulfamethoxazole, tylosin, vancomycin, yrothricin and derivatives, esters, salts and mixtures thereof.

In embodiments, the API comprises an antihistamine selected from the group consisting of chlorcyclizine, diphenhydramine, mepyramine, methapyrilene, tripelennamine and derivatives, esters, salts and mixtures thereof.

In embodiments, the API comprises a corticosteroid selected from the group consisting of alclometasone dipropionate, amcinafel, amcinafide, amcinonide, beclomethasone, beclomethasone dipropionate, betamethsone, betamethasone benzoate, betamethasone dexamethasone-phosphate, dipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, difluorosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradrenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, α-methyl dexamethasone, methylprednisolone, methylprednisolone acetate, mometasone furoate, paramethasone, prednisolone, prednisone, pregnenolone, progesterone, spironolactone, triamcinolone, triamcinolone acetonide and derivatives, esters, salts and mixtures thereof.

In embodiments, the API comprises a hormone selected from the group consisting of methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5a-dihydrotestosterone, testolactone, 17a-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5a-pregnan-3b,20a-diol sulfate, 5a-pregnan-3b,20b-diol sulfate, 5a-pregnan-3b.-ol-20-one, 16,5a-pregnen-3b-ol-20-one, 4-pregnen-20b-ol-3-one-20-sulfate, acetoxypregnenolone, anagestone acetate, cyproterone, dihydrogesterone, fluorogestone acetate, gestadene, hydroxyprogesterone acetate, hydroxymethylprogesterone, hydroxymethyl progesterone acetate, 3-ketodesogestrel, megestrol, melengestrol acetate, norethisterone and derivatives, esters, salts and mixtures thereof.

In embodiments, the API comprises a non-steroidal anti-inflammatory agent selected from the group consisting of azelaic acid, oxicams, piroxicam, isoxicam, tenoxicam, sudoxicam, CP-14,304, salicylates, aspirin, disalcid, benorylate, trilisate, safapryn, solprin, diflunisal, fendosal, acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof.

In embodiments, the API comprises a vasodilator selected from the group consisting of ethyl nicotinate, capsicum extract and derivatives, esters, salts and mixtures thereof. In embodiments, the stent assembly includes a low-bulk mesh jacket designed to promote a stable layer of endothelial cells.

In embodiments, the mesh comprises fiber having a low diameter that allows each endothelial cell to fully cover and overlap each fiber, thereby forming a layer of endothelial cells that adhere to tissue on either side of the fiber. The thus formed endothelial layer is substantially stable with a substantially reduced tendency to break away and form emboli.

In embodiments, the mesh fiber comprises material that encourages adherence of endothelial cells, thereby encouraging endothelial layer stability.

In embodiments, each mesh fiber is spaced a distance from a neighboring fiber thereby preventing a single endothelial cell from adhering to more than one fiber, thereby reducing the chance that endothelial cells will break free of the stent, for example as a result of natural stent pulsation during blood flow.

In embodiments, the stent jacket optionally comprises a mesh that is knitted. In accordance with some embodiments of the present invention, the stent jacket mesh is optionally formed from a single fiber or a single group of fibers.

In embodiments, the stent assembly includes a stent jacket comprising an expansible mesh structure, formed of fibers of a diameter between about 7 micrometers and about 18 micrometers, the diameter having a property of forming a substantially stable layer of endothelial cells, covering the fibers, thus reducing to platelet aggregation, and an expansible stent, operatively associated with the stent jacket.

In embodiments, the fiber diameter is between about 10 micrometers and about 15 micrometers.

In embodiments, the fiber diameter is between about 11 micrometers and about 14 micrometers.

In embodiments, the fiber diameter is between about 12 micrometers and about 13 micrometers.

In embodiments, the fiber diameter is between about 12.5 micrometers. In embodiments, the mesh is formed as a single knit. In embodiments, the fiber is formed from multiple filaments.

In embodiments, the mesh jacket structure comprises a retracted state and a deployed state, and further in the deployed state, the mesh structure defines apertures having a minimum center dimension, which is greater than about 180 micrometers, thus minimizing occurrences of a single endothelial cell adhering to more than one fiber, across one of the apertures, and reducing a chance of endothelial cells breaking free as a result of natural stent pulsation with blood flow.

In embodiments, the minimum center dimension is greater than about 200 micrometers.

According to another aspect of the invention, there is provided a method for manufacturing a stent assembly for expanding in vivo vessel lumens, the method comprising: providing two axially aligned radially expandable mesh stents, comprising a first stent and a second stent, at a predetermined distance from each other, attaching a first end of a stent jacket to the first stent, attaching a second end of the stent jacket to the second stent, such that an intermediate portion of the jacket spans the predetermined distance, and encircling a third stent in a contracted state coaxially aligned within the jacket.

In embodiments the method includes: expanding the two axially radially expandable mesh stent, and angling the third stent at an angle to an axis running between the first stent and the second stent of at least about 15 degrees and no more than about 165 degrees.

In embodiments the method includes: angling a fourth stent at an angle to an axis running between the first stent and the second stent of at least about 15 degrees and no more than about 165 degrees.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1a-1d show deployment of prior art stents in bifurcating vessels;

FIGS. 2a-2e show stents and stent jackets being deployed in cross sections of bifurcating vessels, according to embodiments of the invention; and

FIGS. 3a-8d show alternative embodiments of the stents and stent jackets of FIG. 2e being deployed in cross sections of bifurcating vessels, according to embodiments of the invention.

### DESCRIPTION OF THE PREFERRED EMBODIMENTS

The present invention, which relates to stent assemblies configured for assembling in bifurcating vessels, is herein described, by way of example only, with reference to the accompanying drawings. The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Referring now to the drawings:

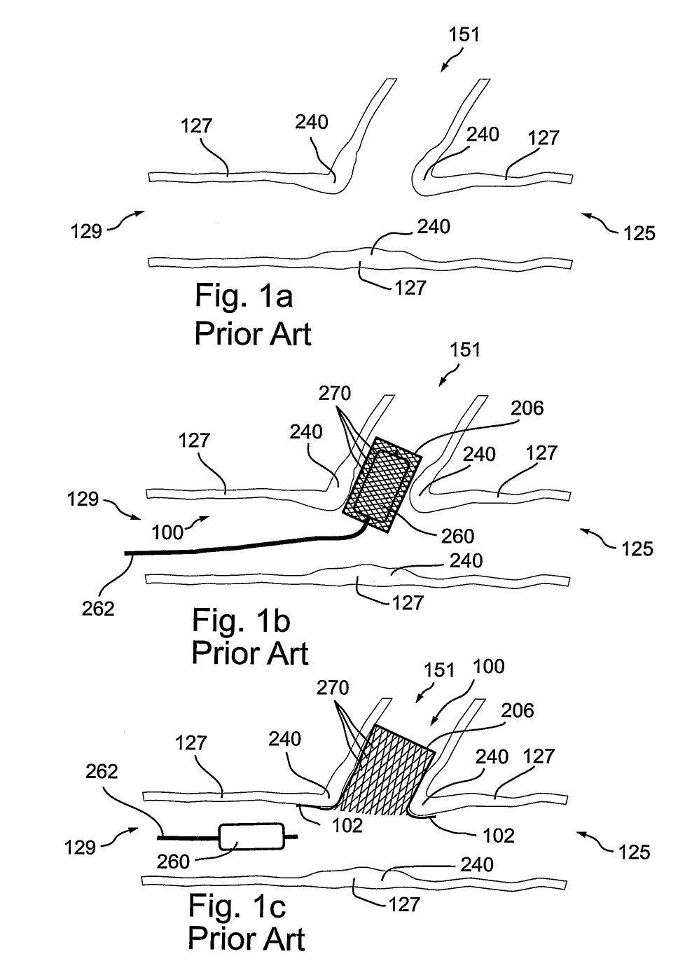

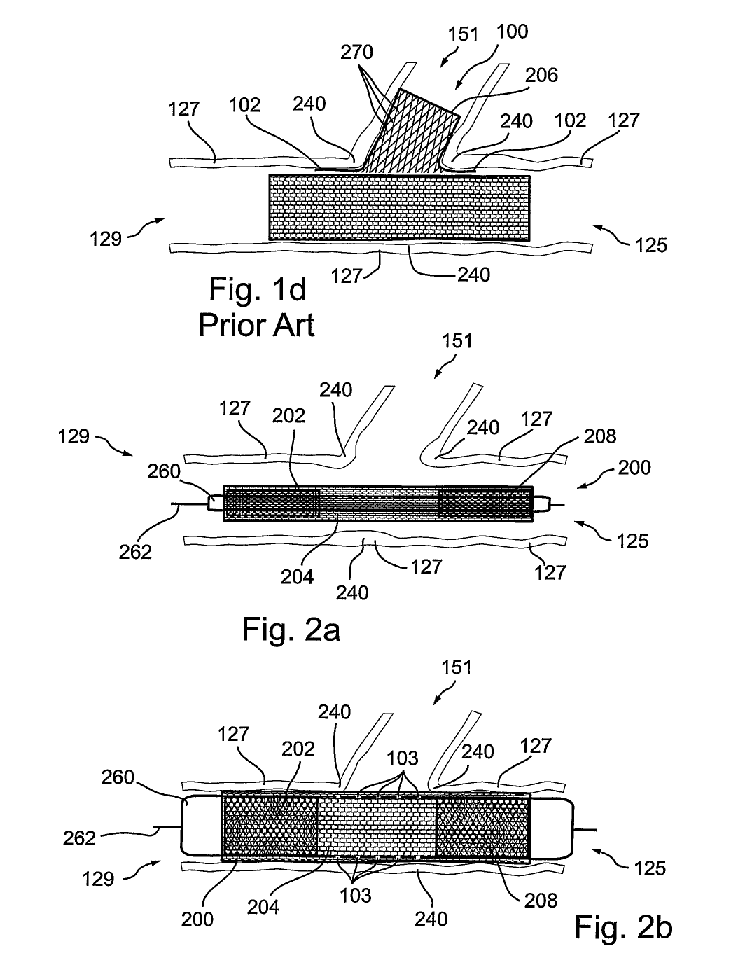

In FIG. 1a, arteries 127 form an upper branch vessel lumen 151, a proximal parent vessel lumen 129 and a distal parent vessel lumen 125.

FIGS. 1b-1d show the crush method, noted above, for treating a bifurcation. As seen in FIG. 1b, a crush stent assembly 100 comprises a branch stent 206 configured for expansion in upper branch lumen 151. Branch stent 206, shown herein without a jacket, comprises a metal or polymer tubular structure having mesh-like, apertures 270. Branch stent 206 is shown encircling a balloon 260 and, upon expansion of balloon 260, branch stent 206 expands radially outward.

As seen in FIG. 1c, branch stent 206 has expanded radially in upper branch lumen 151 so that branch stent 206 presses against a stenotic area of tissue 240, thereby compressing and cracking stenotic area 240 radially outward within upper branch lumen 151. To further ensure flow of blood, a second balloon (not shown) is expanded against a flange 102 to crush flange 102 into proximal lumen 129 and into distal lumen 125.

Deployed stent assembly 100 crushes stenotic tissue 240 in lumens 151, 129 and 125, thereby allowing better circulation through arteries 127. However, as noted above and seen in FIG. 1d, branch stent 206 creates a significant amount of metal related to flange 102 that may subject artery walls 127 to restenosis, in addition to causing turbulence and thrombosis formation.

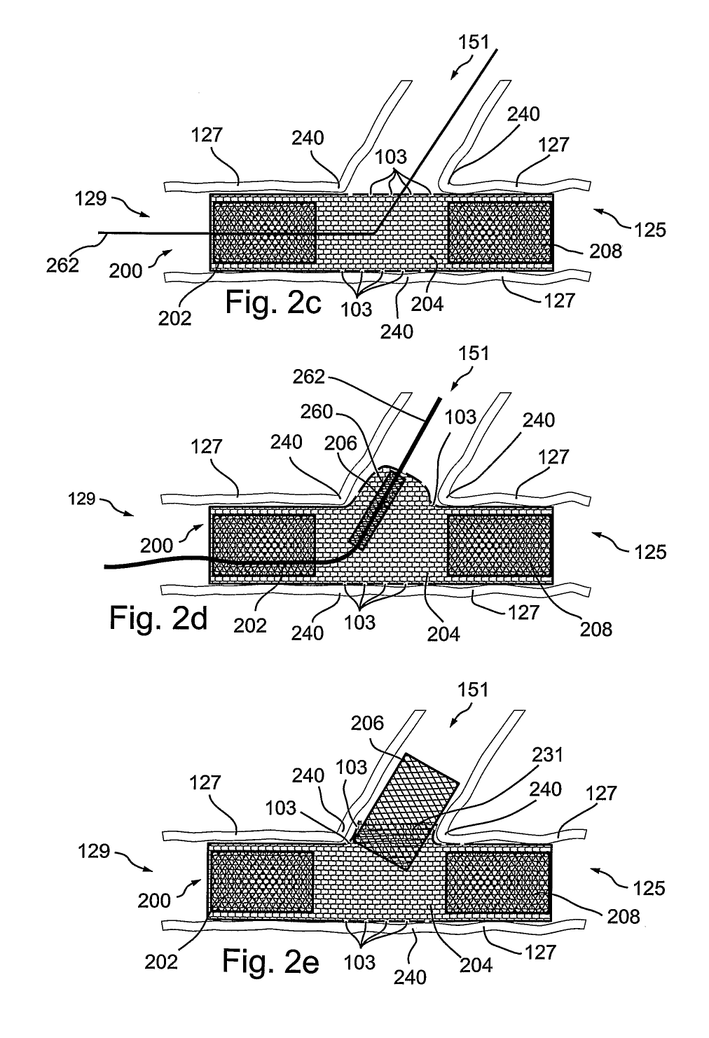

Referring to FIG. 2a, in an embodiment of the present invention, a stent system 200, comprises a proximal parent vessel stent 202 and a distal parent vessel stent 208 that are covered by an external jacket 204. Assembly 200 is positioned in artery 127 so that proximal stent 202 is positioned in proximal lumen 129 and distal stent 208 is positioned in distal lumen 125. In embodiments, for example for use in a coronary vessel, proximal stent 202 is positioned between at least one millimeter and not more than about 20 millimeters from distal stent 208. In other embodiments, proximal stent 202 is positioned about three millimeters from distal stent 208. Optionally, proximal stent 202 and distal stent 208 are placed in positions that stretches external jacket 204 therebetween.

In alternative embodiments proximal stent 202 and distal stent 208 are configured and appropriately sized as cardiovascular stents, peripheral stents, abdominal aortic aneurysm stents, cerebral stents, carotid stents, endovascular stents, aortic valve stents, and pulmonary valve stents.

As seen in FIG. 2b, balloon 260 has been inflated, thereby expanding stents 202 and 208 so that stent jacket 204 spans upper branch lumen 151.

Optionally, balloon 260 is inflated in a manner that crushes stent jacket 204 to aid in opening in lumens 151, 129 and 125 and to avoid jailing of upper branch lumen 151 by stent jacket 204.

As seen in FIG. 2c, balloon 260 has been removed and the structure of stent jacket 204 can be appreciated. Stent jacket 204 typically comprises a knitted material having large apertures 103.

As seen in FIG. 2d, branch stent 206 positioned on balloon 260 has been pressed into stent jacket 204, through one of apertures 103. As seen in FIG. 2e, branch stent 206 has been expanded, thereby expanding aperture 103 and causing an encircling portion of jacket 231 to encircle branch stent 206.

In addition to the support provided by stents 202, 206 and 208, stent jacket 204 spanning therebetween, supports stenotic tissue 240 at the bifurcation of upper branch lumen 151. Using stent jacket 204 as a support along the bifurcation of upper branch lumen 151 results in low bifurcation-related bulk that could cause restenosis and/or thrombosis noted above.

In alternative embodiments, balloon 260 (FIG. 2d) is first used alone to predilate one of apertures 103, after which unexpanded branch stent 206 is pressed through predilated aperture 103 and expanded in upper branch lumen 151.

In embodiments, stents 202, 206 and 208 comprise any metallic base including, inter alia: stainless steel, nitinol, tantalum, MP35N alloy, a cobalt-based alloy, a cobalt-chromium alloy, platinum, titanium, or other biocompatible metal alloys.

In further embodiments, stents 202, 206 and 208 are deployed in any vessel comprising, inter alia: cardiovascular tissue, peripheral tissue, an abdominal aortic aneurysm, cerebral tissue, carotid tissue, endovascular tissue, aortic valves, and/or pulmonary tissue.

In still further embodiments, stent jacket 204 comprises any material manufactured by a process including, inter alia: interlacing knitting, interlocked knitting, braiding, interlacing, and/or dipping a porous mold into one or more reagents.

As used herein, any reference to a “knitted material” includes any material that is manufactured by a knitting process, including, inter alia: a material knitted from a single fiber, similar to the process used in pantyhose nylon; a double fiber knit, referred to as a “double knit material”; and includes fibers, either mono filament or multi filament fiber of, inter alia: polyethylene, polyvinyl chloride, polyurethane, nylon, a biocompatible polymer fiber, and stainless steal nitinol, or any other metal.

In embodiments, proximal stent 202, distal stent 208 and branch stent 206 comprise a metallic base from the group consisting of: stainless steel, nitinol, tantalum, MP35N alloy, a cobalt-based alloy, a cobalt-chromium alloy, platinum, titanium, or other biocompatible metal alloys.

In embodiments, proximal stent 202, distal stent 208 and branch stent 206 are manufactured with sufficient diameters to press at least a portion of the inner walls of artery 127 with a pressure of at least one atmosphere and no more than about 50 atmospheres. In embodiments, proximal stent 202, distal stent 208 and branch stent 206 are manufactured with sufficient diameters to press at least a portion of the inner walls of artery 127 with a pressure of about 15 atmospheres.

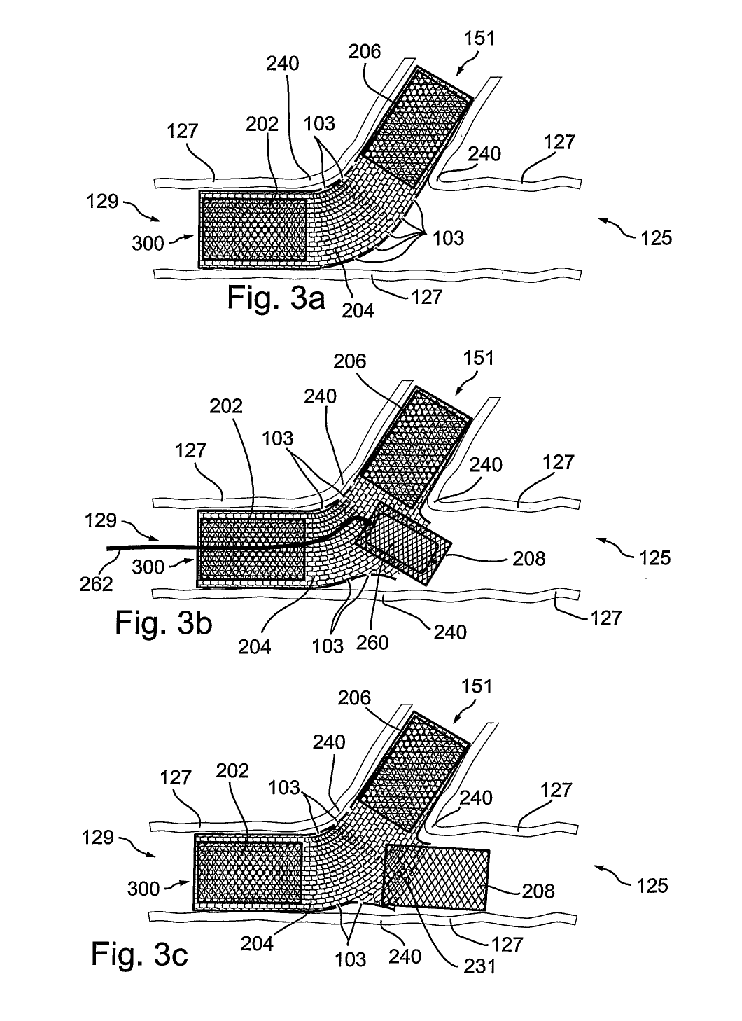

FIG. 3a shows a stent system 300 in which proximal stent 202 has been deployed in proximal lumen 129, and branch stent 206 has been deployed in upper branch lumen 151, while stent jacket 204 spans across distal lumen 125. Typically, upper branch lumen 151 has a smaller diameter than proximal lumen 129 and first balloon (not shown) having a smaller expanded diameter is used to expand branch stent 206.

As seen in FIG. 3b, following expansion of stent 206, a second balloon 260 having a large expanded diameter is used to expand proximal lumen stent 202.

As seen in FIG. 3b, distal parent vessel stent 208 is pushed through apertures 103. As seen in FIG. 3c and distal parent vessel stent 208 has been expanded in distal lumen 125.

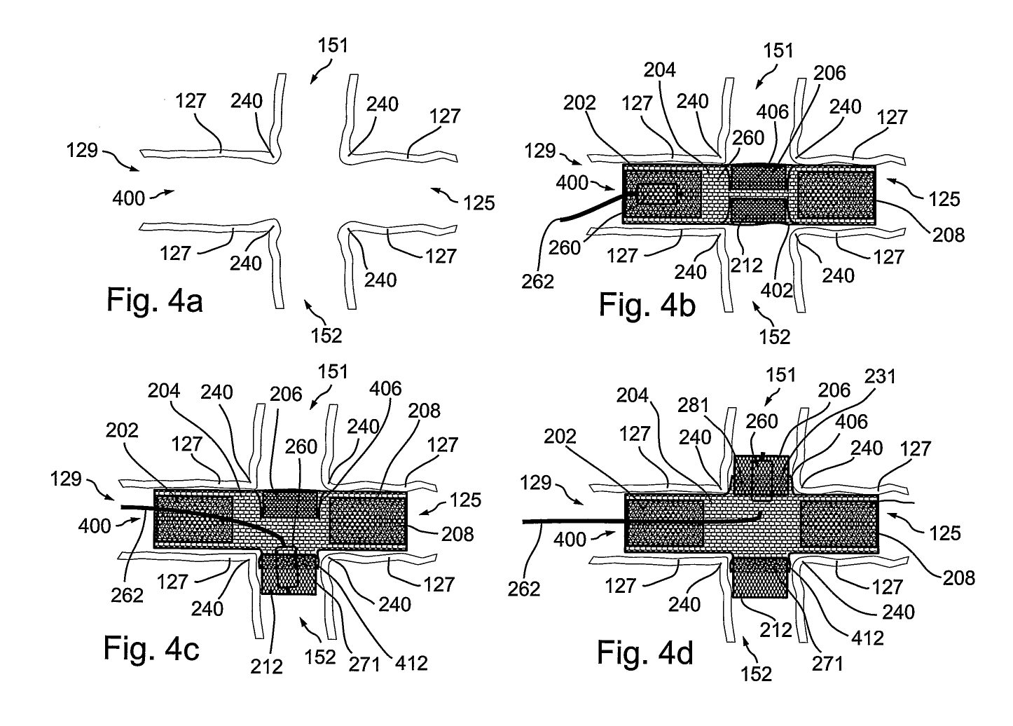

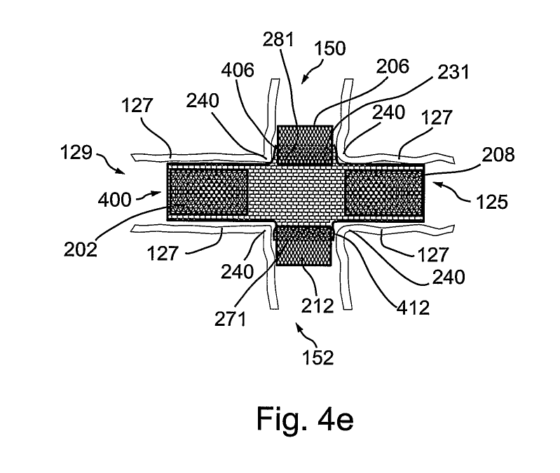

Referring to FIG. 4a, arteries 127 include a lower side branch lumen 152. As seen in FIG. 4b, a dual branch stent assembly 400 comprises stent jacket 204 having an upper sleeve 406 that is partially inside-out and surrounding upper branch stent 206. Stent jacket 204 further comprises a lower sleeve 412 that is inside out and surrounding a lower branch stent 212.

Dual branch stent assembly 400 has been positioned so that distal stent 208, upon expansion with a balloon (not shown), opens distal lumen 125. Proximal stent 202 is then expanded with balloon 260 to open proximal lumen 129.

As seen in FIG. 4c, balloon 260 has been positioned inside lower branch stent 212 and during expansion, balloon 260 is used to push lower branch stent 212 into lower branch lumen 152, thereby straightening lower jacket 204 so that sleeve 412 is no longer inside-out. Balloon 260 then expands lower branch stent 212 to open lower branch lumen 152.

As seen in FIG. 4d, balloon 260 has been positioned inside upper branch stent 206 and, during expansion, balloon 260 is used to push upper branch stent 206 into upper branch lumen 151, thereby straightening upper branch sleeve 406. Balloon 260 then expands upper branch stent 206 to open upper branch lumen 151.

As seen in FIG. 4e, an encircling portion 271 of lower branch sleeve 412, partially covers lower branch stent 212 while an encircling portion 281 of upper branch sleeve 406 partially covers upper branch stent 206, thereby providing support of stenotic tissue 240 therebetween.

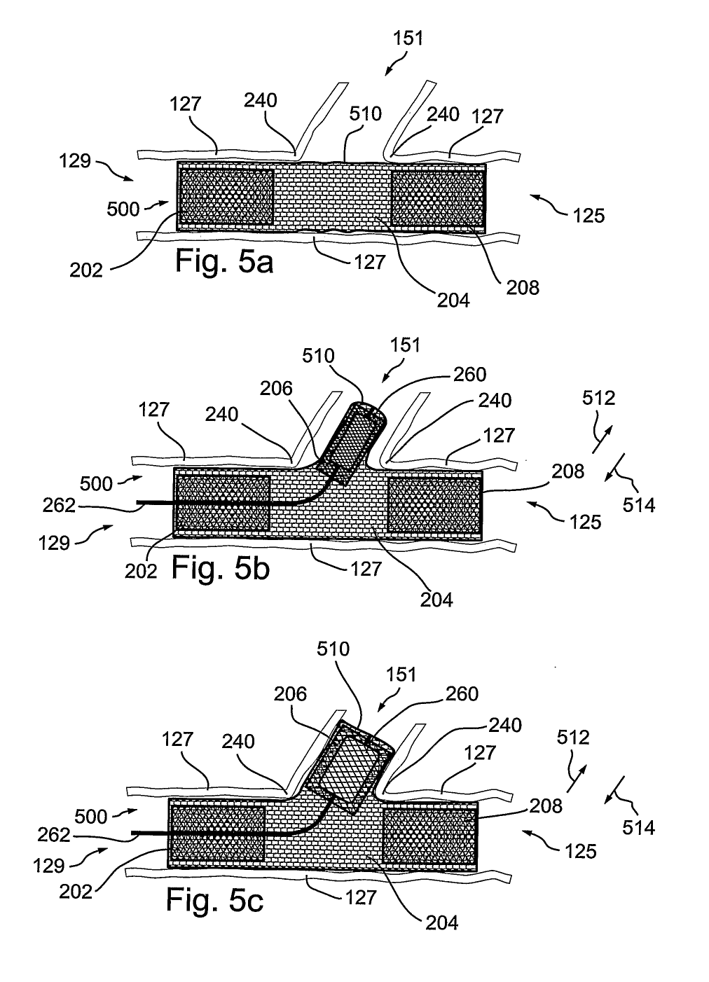

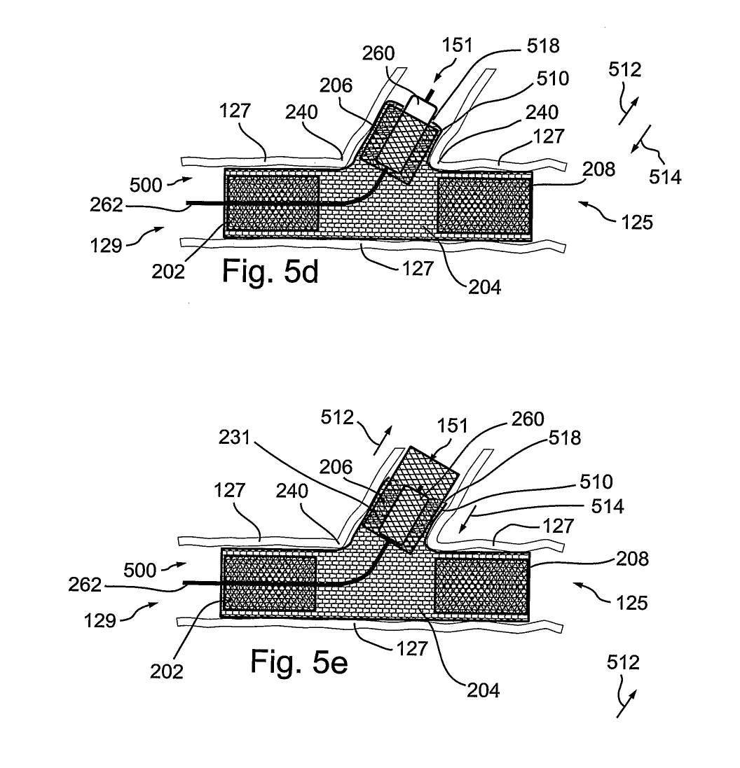

Referring to FIG. 5a, a stent assembly 500 has been positioned and expanded so that proximal stent 202 is positioned in proximal lumen 129 and distal stent 208 is positioned in distal lumen 125. Stent jacket 204, positioned between stents 202 and 208, includes a stretchable material 510. As seen in FIG. 5b, balloon 260, surrounded by unexpanded upper branch stent 206 has been pressed into stretchable material 510, causing stent jacket 204 to bulge into upper branch lumen 151.

In FIG. 5c, balloon 260 has been expanded, thereby causing a partial expansion of upper branch stent 206. Partially expanded upper branch stent 206 stretches stretchable material 510, creating considerable tension on the portion of stent jacket 204 that spans upper branch lumen 151.

In FIG. 5d, balloon 260 has been partially deflated and pressed in an upward direction 512, thereby puncturing material 510 and creating an opening 518. Partially deflated balloon 260 is then moved in a downward direction 514 and partially inflated to expand and be secured within upper branch stent 206. Balloon 260 and upper branch stent 206 are then moved in upward direction 514 causing upper branch stent 206 to pass through opening 518 and into upper branch lumen 151.

Balloon 260 is then fully expanded to cause upper branch stent 206 to fully expand. As seen in FIG. 5e, upper branch stent 206 is partially covered by stretchable material 510, fully expanded in upper branch lumen 151 while balloon 260 has been deflated and is being moved in direction 514 to be removed percutaneously from artery 127.

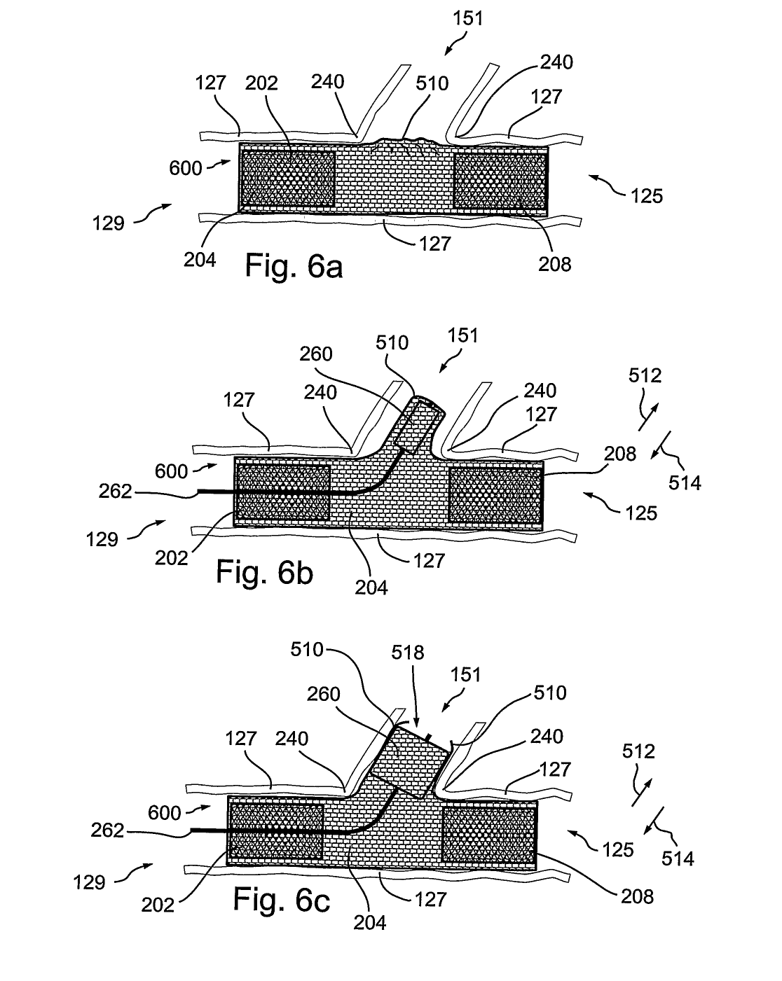

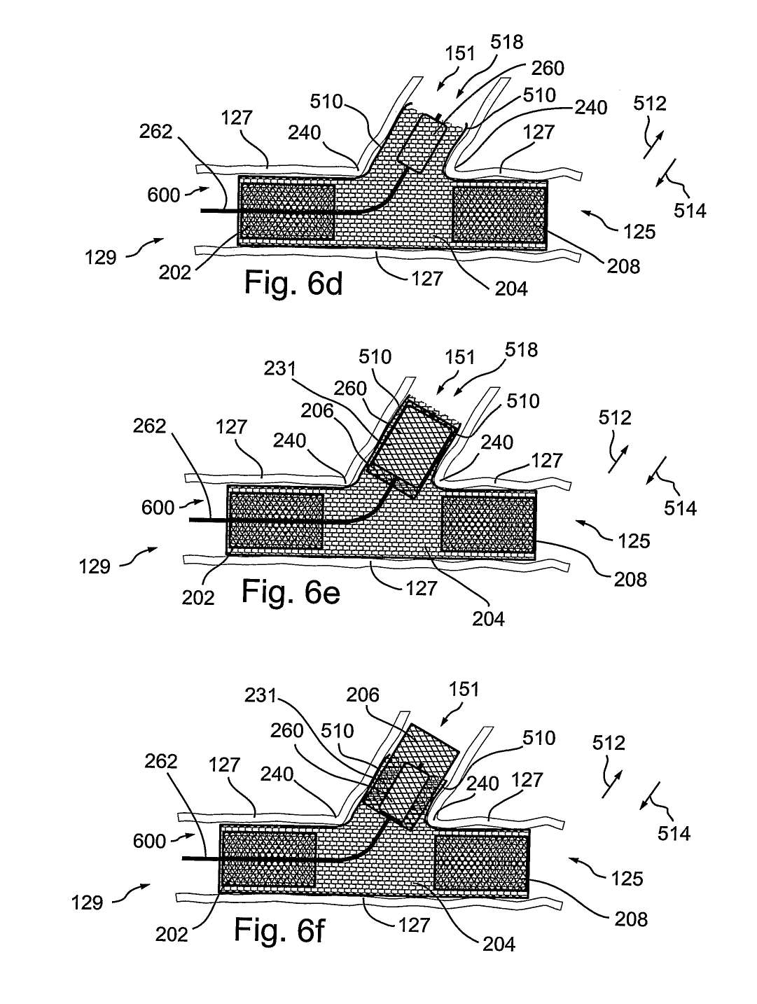

Referring to FIG. 6a, a stretch stent assembly 600 has been positioned and expanded so that proximal stent 202 is positioned in proximal lumen 129 and distal stent 208 is positioned in distal lumen 125. As seen in FIG. 6b, balloon 260, has been pressed into stretchable material 510, causing stent jacket 204 to bulge into upper branch lumen 151.

In FIG. 6c, balloon 260 has been fully expanded, thereby puncturing material 510 and creating opening 518. In FIG. 6d, balloon 260 has been partially deflated and pulled downward in direction 514. Following loading of upper branch stent 206, as seen in FIG. 6e, balloon 260 is partially inflated to move upper branch stent 206 through opening 518. With upper branch stent 206 properly positioned in upper lumen 151, balloon 260 is then fully expanded so that upper branch stent 206 expands to fully open upper branch lumen 151.

Balloon 260 is then deflated and pulled percutaneously in proximal direction 514 and removed from arteries 127. FIG. 6f shows branch stent 206 fully expanded in branch lumen 151 and balloon 260 being removed in direction 514.

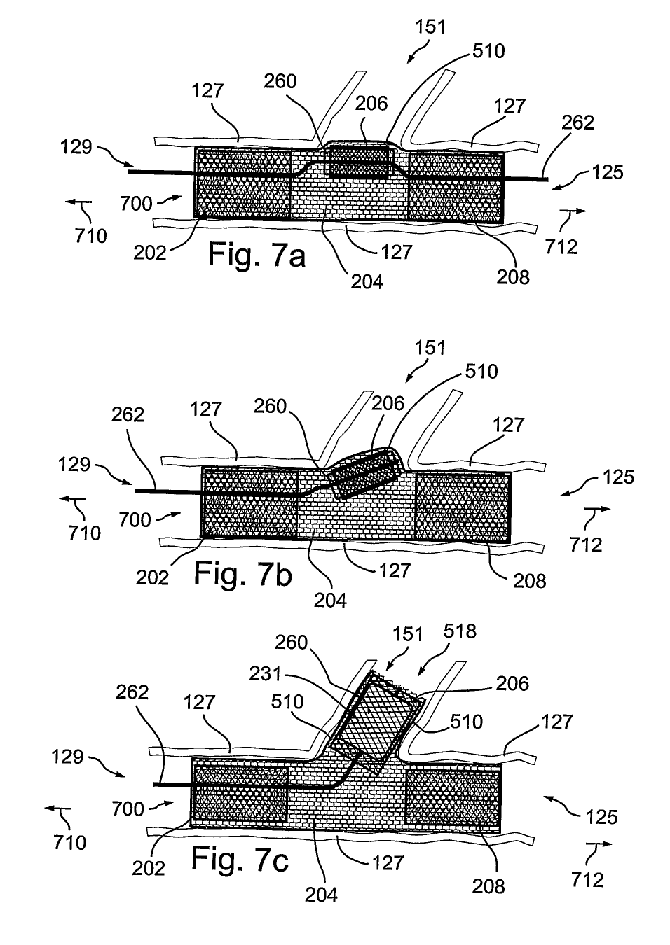

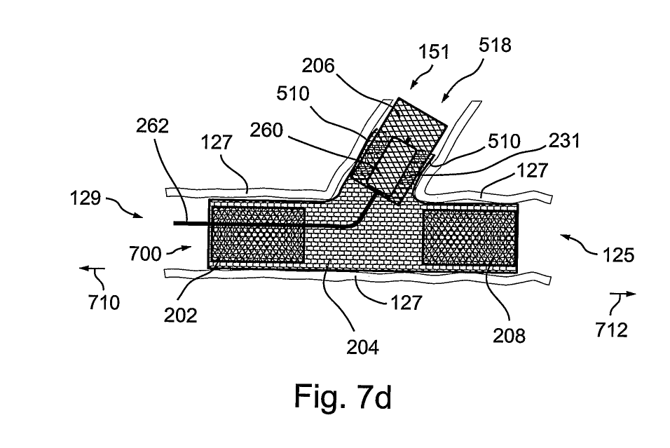

Referring to FIG. 7a, assembly 700 has been positioned and expanded so that proximal stent 202 is positioned in proximal lumen 129 and distal stent 208 is positioned in distal lumen 125. A catheter 262 spans from distal lumen 125 through proximal lumen 129 and is positioned adjacent to upper branch lumen 151 with upper branch stent 206 surrounding balloon 260.

In embodiments, as seen in FIG. 7b, catheter 262 is pulled in a proximal direction 710 until the distal portion of catheter 262 is fully contained within balloon 260. Catheter 262 is then moved in a distal direction 712 to cause stretchable material 510 to bulge into upper branch lumen 151.

As seen in FIG. 7c, balloon 260 has been expanded, thereby expanding upper branch stent 206, piercing material 510 and creating opening 518. As seen in FIG. 7d, balloon 260 has been deflated, leaving upper branch stent 206 partially covered by stent jacket 204.

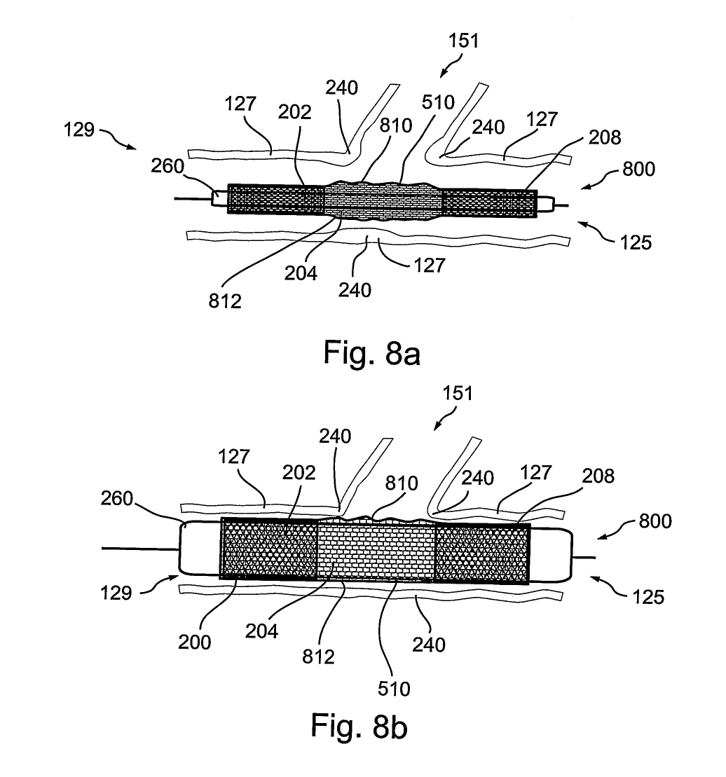

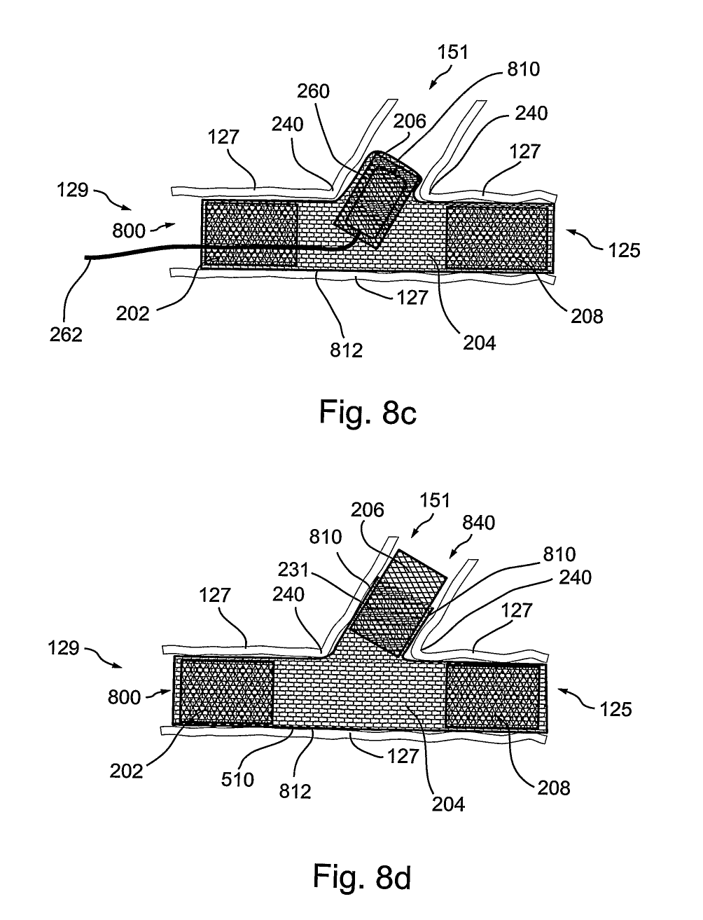

Referring to FIG. 8a, stent system 800 comprises a jacket having billowing walls 812 that include an upper billowing wall potion 810. In embodiments, billing walls include a biocompatible adhesive so that upon inflation, balloon 260 presses billowing wall 812 against artery 127, thereby creating folds in billowing walls 812.

As balloon 260 continues to expand, folds in billowing wall 812 are compressing to adhere to each other and compressed against artery 127. In distinct contrast, as seen in FIG. 8c, upper billowing wall portion 810 is adjacent to upper branch lumen 151, is pressed into branch lumen 151 and does not form adherent folds.

As seen in FIG. 8d further expansion of upper branch stent 206 punctures stent jacket 204, creating a punctured opening 840 and upper branch stent 206 has opened upper branch lumen 151.

As used herein, the terms proximal and proximally refer to a position and a movement in an upstream direction from lumen 129 toward vessel lumen 151. As used herein, the terms distal and distally refer to a position and a movement, respectively, in a downstream direction from lumen 151 toward lumen 129. In embodiments, stent jacket 204 has a thickness of at least about 20 microns and no more than about 200 microns.

It is expected that during the life of a patent maturing from this application many relevant bifurcating stent materials and manufacturing techniques will be developed and the scope of the term bifurcating stent is intended to include all such new technologies a priori.

As used herein the term “about” refers to ±10%

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A stent assembly for expanding in vivo vessels, the assembly comprising:
a) two stents, a first stent and a second stent, said two stents positioned so that a forward end of said first stent is separated by a predetermined distance from a rearward end of said second stent; and
b) a stent jacket spanning said predetermined distance such that a first end of said jacket is operatively associated with said first stent and a second end of said jacket is operatively associated with said second stent.

2. The assembly according to claim 1, wherein upon radial expansion of said first stent and said second stent, said first end of said jacket expands radially encircles at least a portion of said first stent and said second end of said jacket expands radially and encircles at least a portion of said second stent.

3. The assembly according to claim 2, wherein said stent jacket spanning said predetermined distance comprises a length sufficient to longitudinally encircle an axially disposed third stent in a contracted state that is movably set on a guide wire.

4. The assembly according to claim 3, wherein said stent jacket spanning said predetermined distance comprises an internal surface configured to have a cross sectional diameter sufficient to encircle said third stent while said assembly is being delivered to an in situ location.

5. The assembly according to claim 4, wherein said guide wire is configured with an angulated distal portion that allows manipulation proximate to a portion of said stent jacket following expansion of said first stent and said second stent at said in situ location.

6. The assembly according to claim 5, wherein said angulated distal portion comprises an angle to an axis running between said first stent and said second stent of at least about 15 degrees and no more than about 165 degrees.

7. The assembly according to claim 6, said angulated distal portion of said guide wire has a cross-sectional diameter and said stent jacket spanning said predetermined distance comprises at least one aperture of sufficient diameter to allow passage of said cross-sectional diameter.

8. The assembly according to claim 7, wherein said portion of said guide wire is configured with sufficient strength to be manipulated through said aperture.

9. The assembly according to claim 8, wherein said at least one aperture is expandable and configured to expand to a diameter sufficient to encircle an outer surface of said third stent while said third stent in said contracted state.

10. The assembly according to claim 9, wherein the mean diameter of said at least one aperture is configured to further expand when said contracted third stent is expanded while encircled by said aperture.

11. The assembly according to claim 9, wherein at least a portion of said stent jacket spanning said predetermined distance is configured to encircle at least a portion of an outer surface of said third stent when said third stent is in an expanded state.

12. The assembly according to claim 6, wherein a first end of said third stent comprises a friction surface configured to catch a portion of said stent jacket when said friction surface is pressed against said portion while said third stent is expanding.

13. The assembly according to claim 12, wherein said stent jacket comprises a stretchable material configured to stretch across said first end of said stent while said third stent is expanding during said pressing.

14. The assembly according to claim 13, wherein following expansion of said third stent, a stretched portion of said stent jacket is configured to be punctured by a puncturing tool.

15. The assembly according to claim 14, including an intact portion of said stent jacket spanning said predetermined distance, said intact portion configured to remain intact following said puncturing.

16. The assembly according to claim 15, wherein at least a portion of said intact portion includes at least one fold, said at least one fold being adhered by a pressure-sensitive self-adhering adhesive.

17. The assembly according to claim 14, wherein said punctured portion of said stent jacket is expandable and configured to form a mean diameter that is sufficient to allow said third stent to pass through said puncture.

18. The assembly according to claim 14, wherein said puncturing tool includes an expandable balloon.

19. The assembly according to claim 18, wherein said stent jacket spanning said predetermined distance comprises at least one aperture configured to encircle said expandable balloon in a contracted state.

20. The assembly according to claim 19, wherein said at least one aperture is configured to rip as said expandable balloon is inflated.

21. The assembly according to claim 20, wherein a portion of an outer surface of said third stent is configured to slidingly pass through said aperture following said rip and said aperture is configured to remain encircled around at least a portion of an outer surface of said third stent.

22. The assembly according to claim 6, wherein while said assembly is being delivered to an in situ location:
a first portion of said stent jacket spanning said predetermined distance is configured to encircle said axially disposed third stent in a contracted state; and
a second portion of said stent jacket spanning said predetermined distance is configured to encircle an axially disposed fourth stent in a contracted state.

23. The assembly according to claim 22, wherein said guide wire upon which said third stent is set comprises a first guide wire and said fourth stent is axially set on a second guide wire having an angulated distal portion comprising an angle to an axis running between said first stent and said second stent of at least about 15 degrees and no more than about 165 degrees.

24. The assembly according to any one of claims 1-23, wherein following expansion the vessels are supported with one layer of stent metal.

25. A method for manufacturing a stent assembly for expanding in vivo vessel lumens, the method comprising:
a) providing two axially aligned radially expandable mesh stents, comprising a first stent having a forward end at a predetermined distance from a rearward end of a second stent;
b) attaching a first end of a stent jacket to said first stent;
c) attaching a second end of said stent jacket to said second stent, such that an intermediate portion of said jacket spans said predetermined distance; and
d) encircling a third stent in a contracted state coaxially aligned within said jacket.

26. The method according to claim 25, including:
e) encircling a fourth stent in a contracted state coaxially aligned within said jacket.
